(19) United States
(12) Patent Application Publication
(10) Pub. No.: US 2023/0190168 A1
(43) Pub. Date: Jun. 22, 2023

(54) ELECTROCARDIOGRAM PROCESSING APPARATUS AND METHOD

(71) Applicant: ATSENS CO., LTD., Gyeonggi-do (KR)

(72) Inventors: Kab Mun CHA, Seoul (KR); Jong Ook JEONG, Gyeonggi-do (KR)

(73) Assignee: ATSENS CO., LTD., Gyeonggi-do (KR)

(21) Appl. No.: 18/166,770

(22) Filed: Feb. 9, 2023

Related U.S. Application Data
(63) Continuation-in-part of application No. 17/226,680, filed on Apr. 9, 2021.

(51) Int. Cl. A61B 5/346

(52) U.S. Cl. CPC A61B 5/346; A61B 5/72

(57) ABSTRACT

A bio-signal measuring apparatus for detecting signal peaks includes a bio-signal sensing circuit configured to sense an electrocardiogram signal by using an electrode attached to the body of an object. The bio-signal measuring apparatus further includes a processor coupled to the biosignal sensing circuit and operable to receive the electrocardiogram signal from the biosignal sensing circuit. The bio-signal measuring apparatus includes a memory that stores the electrocardiogram signal and stores executable instructions that, when executed by the processor, facilitate performance of operations. The operations include determining a time duration (TD) and determining a peak appearing during the time duration (TD) as a valid peak of an electrocardiogram signal.

### CROSS-REFERENCE TO RELATED APPLICATION

This application is a continuation-in-part of U.S. application Ser. No. 17/226,680 filed Apr. 9, 2021, the disclosure of which is incorporated by reference herein in its entirety.

### BACKGROUND

### 1. Field

One or more embodiments relate to an electrocardiogram processing apparatus and method and more specifically, a bio-signal measuring apparatus and method for determining valid peaks of electrocardiograms without being affected by various types of noise, and a computer program for performing the method, where valid peaks of electrocardiograms may be determined during a threshold time interval signifying validity.

### 2. Description of the Related Art

An existing stand-alone or fixed electrocardiogram measuring apparatus includes attachable electrodes having a large gap (distance) therebetween and measures an electrocardiogram signal of a user while the user maintains a fixed posture. Thus, a change in a baseline of the measured electrocardiogram signal is not great and is stable, and the magnitude of the measured electrocardiogram signal is also large.

In contrast, a wearable patch-type electrocardiogram measuring apparatus includes electrodes having a short distance therebetween and frequently causes movement of a user. In an electrocardiogram signal measured in the above way, various types of noise are generated due to the occurrence of movement in addition to general human body noise. Also, a baseline of the electrocardiogram signal measured in the above way is not stable and significantly changes. In addition, the magnitude of the electrocardiogram signal measured in the above way is also relatively smaller than the magnitude of an electrocardiogram signal measured by a high-priced stand-alone or fixed electrocardiogram measuring apparatus.

Accordingly, it is difficult to detect an R-peak of an electrocardiogram signal by using a patch-type electrocardiogram measuring apparatus.

### SUMMARY

The present disclosure provides a wearable device, a method and software of processing an electrocardiogram signal which is capable of accurately determining valid peaks of the electrocardiogram signal including an R-peak of the electrocardiogram signal and solving the technical problems described above.

According to one or more embodiments of the present disclosure, a bio-signal measuring apparatus for detecting signal peaks includes a bio-signal sensing circuit configured to sense an electrocardiogram signal by using an electrode attached to the body of an object. The bio-signal measuring apparatus further includes a processor coupled to the biosignal sensing circuit and operable to receive the electrocardiogram signal from the biosignal sensing circuit. The bio-signal measuring apparatus includes a memory that stores the electrocardiogram signal and stores executable instructions that, when executed by the processor, facilitate performance of operations. The operations include determining a time duration (TD) and determining a peak appearing during the time duration (TD) as a valid peak of an electrocardiogram signal.

One or more embodiments include a bio-signal measuring apparatus for detecting signal peaks by using a complexity value of a signal around a peak, a method of detecting signal peaks in an electrocardiogram, and a computer program for performing the methods.

One or more embodiments include a bio-signal measuring apparatus for detecting, via machine learning or an artificial intelligence algorithm, signal peaks in a time interval having a complexity value exceeding a determined threshold complexity level, a method of detecting signal peaks in an electrocardiogram, and a computer program for performing the methods.

Additional aspects will be set forth in part in the description which follows and, in part, will be apparent from the description, or may be learned by practice of the presented embodiments of the disclosure.

According to one or more embodiments, a bio-signal measuring apparatus for detecting signal peaks includes: a bio-signal sensing circuit configured to sense an electrocardiogram signal by using an electrode attached to the body; a first filtering unit configured to filter the electrocardiogram signal into a first frequency band; a second filtering unit configured to filter the electrocardiogram signal into a second frequency band; a candidate peak detector configured to detect first candidate peaks in a first filtering signal filtered by the first filtering unit and detect second candidate peaks in a second filtering signal filtered by the second filtering unit; a peak integrator configured to define windows by time intervals and calculate, as average peak time values, average values of time values of the first candidate peaks and the second candidate peaks included in the windows; and a valid peak detector configured to calculate, in the first filtering signal and the second filtering signal, complexity values for time intervals including the average peak time values, determine a peak occurrence time value on the basis of the complexity value in the first filtering signal and the complexity value in the second filtering signal, and detect valid peaks in the electrocardiogram signal by using the peak occurrence time value.

In at least one variant, the valid peak detector may define a first time interval including a first average peak time value from among the average peak time values, calculate a first complexity value and a second complexity value in the first filtering signal and the second filtering signal, respectively, within the first time interval and, when both the first complexity value and the second complexity value exceed a preset threshold complexity level, determine, as a peak occurrence time value, the first average peak time value included in the first time interval.

In another variant, the preset threshold complexity level may be a value that is determined on the basis of complexity values of time intervals including R peaks designated in measured electrocardiogram signals.

In further another variant, the complexity values may be values that are calculated on the basis of an occurrence frequency of signals for each section of measured values.

In another variant, when a first window defined by a time interval does not include candidate peaks, the peak integrator may be implemented not to calculate an average peak time value corresponding to the first window.

According to one or more embodiments, a method of detecting signal peaks includes: sensing an electrocardiogram signal by using an electrode attached to the body; filtering the electrocardiogram signal into a first frequency band; filtering the electrocardiogram signal into a second frequency band; detecting first candidate peaks in a first filtering signal generated by the filtering and detecting second candidate peaks in a second filtering signal generated by the filtering; defining windows by time intervals and calculating, as average peak time values, average values of time values of the first candidate peaks and the second candidate peaks included in the windows; and calculating, in the first filtering signal and the second filtering signal, complexity values for time intervals including the average peak time values, determining a peak occurrence time value on the basis of the complexity value in the first filtering signal and the complexity value in the second filtering signal, and detecting valid peaks in the electrocardiogram signal by using the peak occurrence time value.

In at least one variant, the detecting of the valid peaks may include: defining a first time interval including a first average peak time value from among the average peak time values, calculating a first complexity value and a second complexity value in the first filtering signal and the second filtering signal, respectively, within the first time interval, and, when both the first complexity value and the second complexity value exceed a preset threshold complexity level, determining, as a peak occurrence time value, the first average peak time value included in the first time interval.

According to one or more embodiments, a computer program may be stored on a medium to perform, by using a computer, any one of methods according to one or more embodiments.

According to one or more embodiments of the present disclosure, a wearable device includes a biosignal sensing circuit including a plurality of electrodes positioned at a plurality of different locations on a body of an object and configured to sense an electrocardiogram signal and convert the electrocardiogram signal digitally, a processor coupled to the biosignal sensing circuit and operable to receive the electrocardiogram signal from the biosignal sensing circuit, and a memory that stores the electrocardiogram signal and stores executable instructions that, when executed by the processor, facilitate performance of operations. The operations includes (i) generating a first filtered signal by filtering the electrocardiogram signal in a first frequency band, (ii) generating a second filtered signal by filtering the electrocardiogram signal in a second frequency band, (iii) detecting a plurality of peaks in the first filtered signal as first candidate peaks based on a magnitude of the first filtered signal, (iv) detecting a plurality of peaks in the second filtered signal as second candidate peaks based on a magnitude of the second filtered signal, (v) during each preset time window having a predetermined time interval, determining an average peak time value based on a first candidate peak in the first filtered signal and a second candidate peak in the second filtered signal; (vi) determining a time duration (TD) based on the average peak time value; (vii) determining a first complexity value of the first filtered signal and a second complexity value of the second filtered signal in the time duration (TD), the first complexity value and the second complexity value indicative of an occurrence frequency pattern of the plurality of peaks in the first filtered signal and in the second filtered signal, respectively; (viii) determining whether or not both the first complexity value and the second complexity value exceed a preset threshold complexity level, and (ix) upon determination that both the first complexity value and the second complexity value exceed the preset threshold complexity level, determining a peak appearing during the time duration (TD) as a valid peak of the electrocardiogram signal. The wearable device further includes a display coupled to the processor and operable to display the electrocardiogram signal having the valid peak and disregard an electrocardiogram signal having a peak that does not appear during the time duration (TD).

According to one or more embodiments of the present disclosure, a non-transitory computer-readable recording medium storing therein executable instructions that, when executed by a processing system including a processor, facilitate performance of operations as described above.

In at least one variant, the first or the second complexity value is calculated by one of Shannon entropy, Sample entropy, and Turning point ratio.

In another variant, the operations further include determining the preset threshold complexity level based on complexity values of time intervals including R peaks of electrocardiogram signals measured in the past.

In another variant, the determining the first complexity value and the second complexity value further comprises determining the first complexity value or the second complexity value based on the occurrence frequency pattern of each magnitude section of the sensed electrocardiogram signal.

In another variant, the operations further comprise forgoing to determine the average peak time value when no candidate peak is present during a preset time window.

According to one or more embodiments of the present disclosure, a wearable device includes a biosignal sensing circuit including a plurality of electrodes positioned at a plurality of different locations on a body of an object and configured to sense an electrocardiogram signal and convert the electrocardiogram signal digitally, a processor coupled to the biosignal sensing circuit and operable to receive the electrocardiogram signal from the biosignal sensing circuit, a signal processing unit coupled to the processor, and a display coupled to the processor and operable to display the electrocardiogram signal. The signal processing unit includes a first filtering unit and a second filtering unit, a candidate peak detector coupled to the first filtering unit and the second filtering unit, a peak integrator coupled to the candidate peak detector, a valid peak detector coupled to the peak integrator, and output mean coupled to the valid peak detector. The signal processing unit, when executed by the processor, facilitate performance of operations, the operations including (i) generating, with the first filtering unit, a first filtered signal by filtering the electrocardiogram signal in a first frequency band, (ii) generating, with the second filtering unit, a second filtered signal by filtering the electrocardiogram signal in a second frequency band, (iii) detecting, with the candidate peak detector, a plurality of peaks in the first filtered signal as first candidate peaks based on a magnitude of the first filtered signal, (iv) detecting, with the candidate peak detector, a plurality of peaks in the second filtered signal as second candidate peaks based on a magnitude of the second filtered signal, (v) during each preset time window having a predetermined time interval, determining, with the peak integrator, an average peak time value based on a first candidate peak in the first filtered signal and a second candidate peak in the second filtered signal, (vi) determining, with the valid peak detector, a time duration (TD) based on the average peak time value, (vii) determining, with the valid peak detector, a first complexity value of the first filtered signal and a second complexity value of the second filtered signal in the time duration (TD), the first complexity value and the second complexity value indicative of an occurrence frequency pattern of the plurality of peaks in the first filtered signal and in the second filtered signal, respectively; (viii) determining, with the valid peak detector, whether or not both the first complexity value and the second complexity value exceed a preset threshold complexity level, and (ix) upon determination that both the first complexity value and the second complexity value exceed the preset threshold complexity level, determining, with the valid peak detector, a peak appearing during the time duration (TD) as a valid peak of the electrocardiogram signal. The wearable device further includes a display coupled to the processor and operable to display the electrocardiogram signal having the valid peak and disregard an electrocardiogram signal having a peak that does not appear during the time duration (TD).

According to one or more embodiments of the present disclosure, a method includes (i) sensing an electrocardiogram signal by a bio-signal sensing circuit of the bio-signal measuring apparatus, (ii) converting digitally the electrocardiogram signal by the bio-signal sensing circuit, (iii) storing the electrocardiogram signal in a memory by a processor of a bio-signal measuring apparatus, (iv) generating, by the processor, a first filtered signal by filtering the electrocardiogram signal in a first frequency band, (v) generating, by the processor, a second filtered signal by filtering the electrocardiogram signal in a second frequency band, (vi) detecting, by the processor, a plurality of peaks in the first filtered signal as first candidate peaks based on a magnitude of the first filtered signal, (vii) detecting, by the processor, a plurality of peaks in the second filtered signal as second candidate peaks based on a magnitude of the second filtered signal, (viii) during each preset time window having a predetermined time interval, determining, by the processor, an average peak time value based on a first candidate peak in the first filtered signal and a second candidate peak in the second filtered signal; (ix) determining, by the processor, a time duration (TD) based on the average peak time value; (x) determining, by the processor, a first complexity value of the first filtered signal and a second complexity value of the second filtered signal in the time duration (TD), the first complexity value and the second complexity value indicative of an occurrence frequency pattern of the plurality of peaks in the first filtered signal and in the second filtered signal, respectively; (xi) determining, by the processor, whether or not both the first complexity value and the second complexity value exceed a preset threshold complexity level, and (xii) upon determination that both the first complexity value and the second complexity value exceed the preset threshold complexity level, determining, by the processor, a peak appearing during the time duration (TD) as a valid peak of the electrocardiogram signal, (xiii) generating, by the processor, output data displaying information on the electrocardiogram signal having the valid peak, and (xiv) storing, by the processor, the output data in the memory.

In at least one variant, the method further includes transmitting the output data to an external device which displays the electrocardiogram signal having the valid peak based on the output data.

In another variant, the first or the second complexity value is calculated by one of Shannon entropy, Sample entropy, Turning point ratio.

In another variant, the method further includes determining the preset threshold complexity level based on complexity values of time intervals including R peaks of electrocardiogram signals measured in the past.

In another variant, the determining the first complexity value and the second complexity value further comprises determining the first complexity value or the second complexity value based on the occurrence frequency pattern of each magnitude section of the sensed electrocardiogram signal.

In another variant, the method further includes forgoing to determine the average peak time value when no candidate peak is present during a preset time window.

In addition, other methods and other systems for implementing one or more embodiments, and computer-readable recording media recording thereon a computer program for executing the method may be further included.

Other aspects, features, and advantages than those described above will become apparent from the following drawings, claims, and detailed description.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a diagram for explaining an example of use of a bio-signal measuring apparatus 100 according to one or more embodiments of the present disclosure.

FIG. 2 is a diagram for explaining an example of use of the bio-signal measuring apparatus 100 according to one or more embodiments of the present disclosure.

FIG. 3 is a diagram for explaining an example of use of the bio-signal measuring apparatus 100 according to one or more embodiments of the present disclosure.

FIG. 4A illustrates one example of attachment configurations of electrocardiogram electrodes for use with the bio-signal measuring apparatus 100.

FIG. 4B is an exemplary diagram of electrocardiogram signals measured using the electrocardiogram electrodes as shown in FIG. 4A.

FIG. 5A is a block diagram of a bio-signal measuring apparatus 100 according to one or more embodiments of the present disclosure;

FIG. 5B is a block diagram illustrating filtering signal divided into windows according to one or more embodiments of the present disclosure;

FIG. 6 is a block diagram of a bio-signal measuring apparatus 100′ according to one or more embodiments of the present disclosure;

FIG. 7 is a flowchart of a method of detecting signal peaks according to one or more embodiments of the present disclosure;

FIG. 8A is an example view illustrating a first filtering signal FD1;

FIG. 8B is an example view illustrating a second filtering signal FD2;

FIG. 8C is an example view illustrating a third filtering signal FD3;

FIG. 9A is an example view illustrating first candidate peaks CP1;

FIG. 9B is an example view illustrating second candidate peaks CP2;

FIG. 9C is an example view illustrating third candidate peaks CP3;

FIG. 10A is an exemplary diagram of candidate peaks detected according to one or more embodiments of the present disclosure;

FIG. 10B is a view for explaining a process of determining an average peak time value, according to one or more embodiments of the present disclosure;

FIG. 11A is a diagram of a signal filtered to a first frequency band according to one or more embodiments of the present disclosure;

FIG. 11B is a diagram of a signal filtered to a second frequency band according to one or more embodiments of the present disclosure;

FIG. 11C is a diagram of a signal filtered to a third frequency band according to one or more embodiments of the present disclosure;

FIG. 12A is an example view illustrating an electrocardiogram signal; and

FIG. 12B is an example view illustrating an electrocardiogram signal from which signal peaks are detected, according to one or more embodiments.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, the configuration and operation of the present disclosure will be described in detail with reference to embodiments of the present disclosure shown in the accompanying drawings.

While example embodiments are capable of various modifications and alternative forms, embodiments thereof are shown by way of example in the drawings and will herein be described in detail. The effects and features of the present disclosure and the accompanying methods thereof will become apparent from the following description of the embodiments, taken in conjunction with the accompanying drawings. However, the present disclosure is not limited to the embodiments described below and may be embodied in various modes.

Hereinafter, embodiments of the present disclosure will be described in detail with reference to the accompanying drawings, and, when describing with reference to the drawings, the same or corresponding elements are given the same reference numerals and redundant descriptions thereof will be omitted.

In the following embodiments, the terms first, second, etc. may not be used in a limited sense but may be used to distinguish one element or component from another element or component.

The singular forms are intended to include the plural forms as well unless the context clearly indicates otherwise.

The terms “comprises,” “includes,” “have,” etc. when used herein, specify the presence of stated features, elements, and/or components, but do not preclude the presence or addition of one or more other features, elements, or components.

In the drawings, elements or components may be exaggerated or reduced in sizes thereof for convenience of description. For example, the size and thickness of each element or component shown in the drawings are arbitrarily shown for convenience of description, so the present disclosure is not necessarily limited to what is shown.

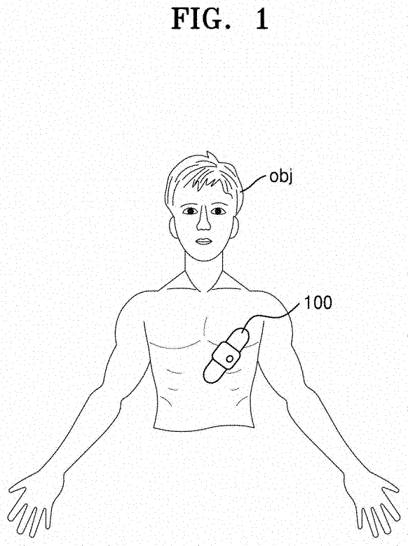

When a certain embodiment is capable of being implemented differently, a particular process order may be performed differently from the described order. For example, two processes described in succession may be performed substantially simultaneously or may be performed in an order opposite to the described order. FIG. 1 is a diagram for explaining an example of use of the bio-signal measuring apparatus 100.

As shown in FIG. 1, the bio-signal measuring apparatus 100 is a device non-invasively or invasively mounted on an object obj to sense an electrocardiogram according to the heartbeat of the object. Here, the object obj may be a body part of a human or animal, such as a human, animal, or chest, but is not limited thereto. Also, an electrocardiogram refers to a graph recording a potential change appearing on a body surface according to a mechanical activity of a heartbeat, such as myocardial contraction/expansion. The meaning of ‘detecting the electrocardiogram’ may mean ‘detecting an electric potential’ generated on the body surface according to the heartbeat of the subject. In some embodiments, the bio-signal measuring apparatus may transmit/receive data with an external device using a communication module. The communication module may include various communication modules such as a wireless Internet module, a short-distance communication module, and a mobile communication module.

The wireless Internet module is connected to an external network according to communication protocols such as wireless LAN (WLAN), Wi-Fi, Wibro (Wireless broadband), Wimax (World Interoperability for Microwave Access), HSDPA (High Speed Downlink Packet Access), etc. means a module that is connected to and performs communication.

The short-range communication module communicates with external devices located in a short distance according to short-range communication methods such as Bluetooth, Radio Frequency Identification (RFID), Infrared Data Association (IrDA), Ultra-Wideband (UWB), and ZigBee. It means a module for performing communication.

The mobile communication module refers to a module that performs communication by accessing a mobile communication network according to various mobile communication standards such as 3rd generation (3G), 3rd generation partnership project (3GPP), and long term evolution (LTE).

However, the present disclosure is not limited thereto, and if various signals and data can be transmitted and received while communicating with the bio-signal measuring apparatus 100, the communicator 140, 140′ may employ a communication module of a different type other than the above-described ones.

The bio-signal measuring apparatus 100 may further include a band-type or gel-type mounting part. The mounting portion may be provided with a flexible material that can be deformed according to the curved surface of the body surface, for example, elastic, that is, stretchable fabric. The mounting portion may be provided in a tape or gel type patch type or wearable type. It is only necessary to be able to sense potential generated on the body surface by being in contact with the body surface of the target object due to being worn through the wearing part.

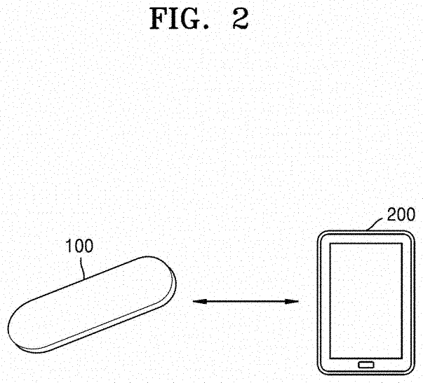

FIG. 2 is a diagram illustrating exemplary operations of the bio-signal measuring apparatus 100.

As shown in FIG. 2, the bio-signal measuring apparatus 100 may operate while exchanging data with the user terminal 200. Here, the user terminal 200 includes a mobile phone, a smart phone, a laptop computer, a digital broadcasting terminal, a personal digital assistant (PDA), a portable multimedia player (PMP), a navigation device, and an MP3 player. It can be used for small electronic devices such as electric toothbrushes, electronic tags, lighting devices, remote controls, fishing rods, etc., but it is not limited thereto, and mobile devices are sufficient, and the terms terminal or device may be used interchangeably.

The bio-signal measuring apparatus 100 may transmit the measured electrocardiogram signal to the user terminal 200. The bio-signal measuring apparatus 100 may transmit data generated based on the measured electrocardiogram signal to the user terminal 200. Here, the generated data includes data obtained by converting the ECG signal into a frequency dimension, a signal obtained by filtering the ECG signal into a predetermined frequency band, data including the ECG signal and measured time values, information on candidate peaks, and valid peaks.

The user terminal 200 communicating with the bio-signal measuring apparatus 100 may be registered through a predetermined registration process and may be provided singly or in plurality.

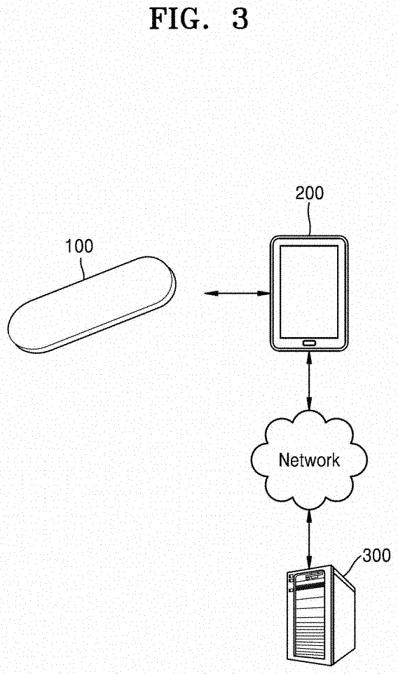

FIG. 3 is another diagram illustrating exemplary operations of the bio-signal measuring apparatus 100.

As shown in FIG. 3, the bio-signal measuring apparatus 100 may operate while exchanging data with the user terminal 200 and/or a data server 300.

The data server 300 stores electrocardiogram signal data received from the plurality of bio-signal measuring apparatus 100 and/or data converted to a frequency dimension, a signal filtered into a predetermined frequency band, an electrocardiogram signal, and measured time values. Including data, information on candidate peaks, information on effective peaks, etc. can be managed.

The data server 300 may manage electrocardiogram data from the bio-signal measuring apparatus 100, candidate peaks in the electrocardiogram signal, and/or information on effective peaks in association with the object. The data server 300 may store electrocardiogram data of the first object in relation to the first object. The data server 300 manages the electrocardiogram signal of the subject.

The data server 300 may perform a function of distributing collected electrocardiogram signals and/or information on candidate peaks and valid peaks in the electrocardiogram signals.

The data server 300 may be designed to provide data, such as electrocardiogram signals measured for the first object and peak information on the electrocardiogram signals, to a user having authority for the first object. A user having authority over the first object may be at least one of a medical worker, a legal person in charge, and a person employed for management. The data server 300 may transmit electrocardiogram data of a subject requested by a medical practitioner.

The data server 300 may generate output data based on data received from the bio-signal measuring apparatus 100. The data server 300 may generate output data based on information about a valid peak of an electrocardiogram signal. The data server 300 may receive measurement data of the bio-signal measuring apparatus 100 through the user terminal 200.

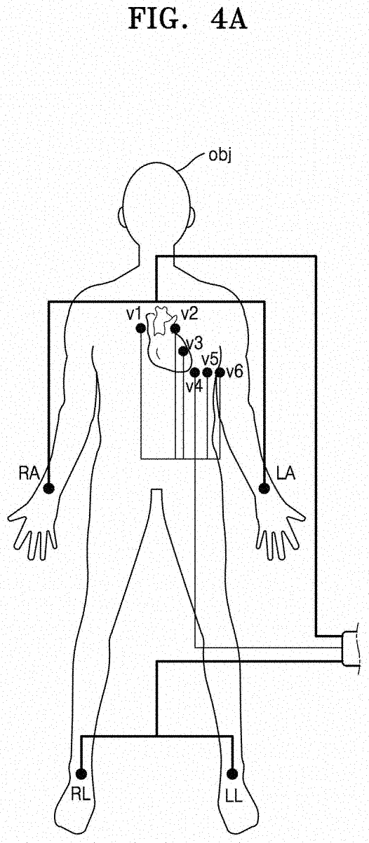

FIG. 4A illustrates one example of attachment configuration of electrocardiogram electrodes available in the pertinent art.

As shown in FIG. 4A, electrocardiogram signals may be measured by attaching ten (10) electrodes to the body of the object obj. A number of electrodes may vary and is not limited to a particular count. The ten (10) electrodes may include limb electrodes RA, LA, RL, and LL and anterior chest electrodes V1, V2, V3, V4, V5, and V6. As shown in the figure, the limb electrodes RA, LA, RL, and LL are attached to the left hand, right hand, left foot, and right foot, respectively, to measure signals. The anterior chest electrodes V1, V2, V3, V4, V5, and V6 may be attached to predetermined positions from the center of the chest to the armpit to measure signals.

FIG. 4B is an exemplary diagram of electrocardiogram signals measured by using the electrocardiogram electrodes as shown in FIG. 4A.

Signal S1 is a signal measured corresponding to the potential difference between RA and LA (e.g., a right hand and a left hand). Signal S2 is a signal measured corresponding to the potential difference between RA and LL (e.g., a right hand and a left foot). Signal S3 is a signal measured corresponding to the potential difference between LA and LL (e.g., a left hand and a left foot). Signal S4 is a signal measured in response to a potential difference between the center of the heart and RA (e.g., a right hand). Signal S5 is a signal measured in response to a potential difference between the center of the heart and the LA (e.g., a left hand). Signal S6 is a signal measured in response to a potential difference between the center of the heart and LL (e.g., a left foot). Signal S7 is the signal measured at electrode V1. Signal S8 is the signal measured at electrode V2. Signal S9 is the signal measured at electrode V3. Signal S10 is a signal measured at electrode V4. Signal S11 is a signal measured at electrode V5. Signal S12 is a signal measured at electrode V6. As illustrated in FIG. 4B, information related to a waveform of a heart and a signal peak is acquired using twelve (12) signals.

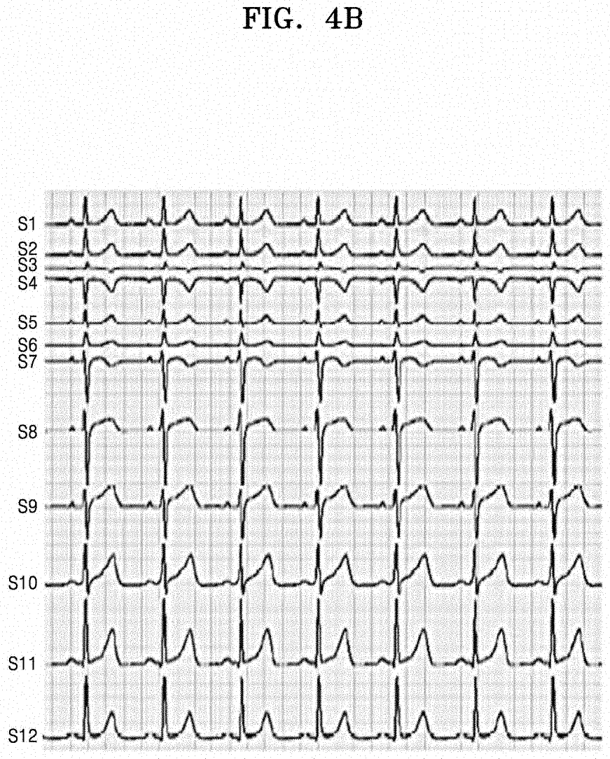

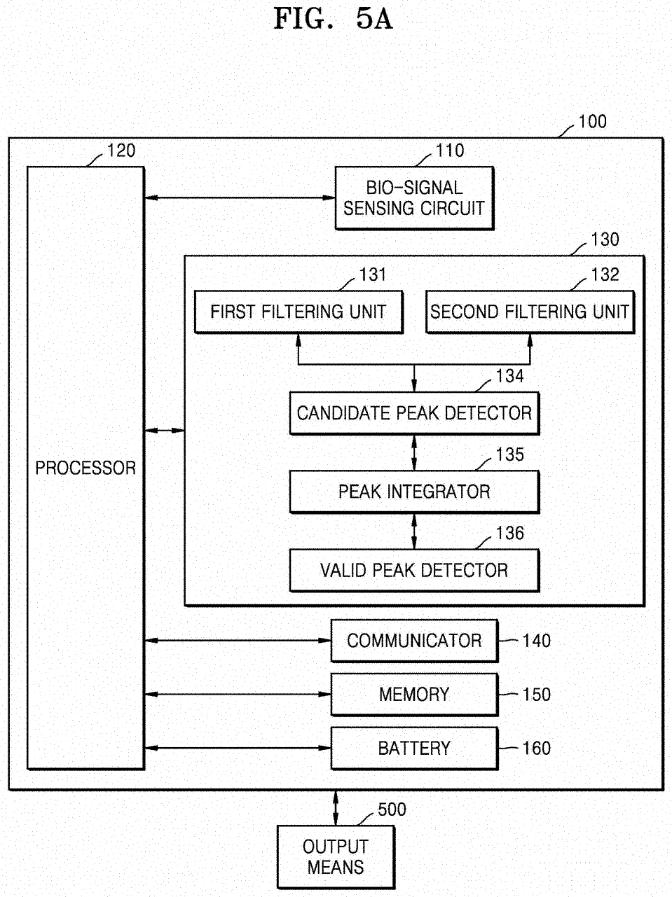

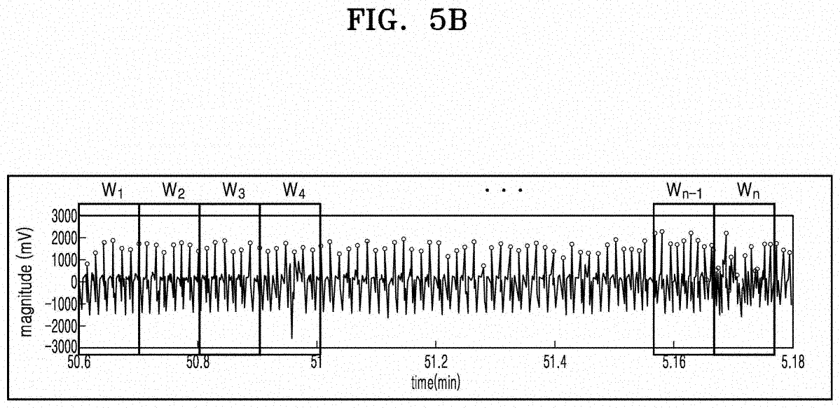

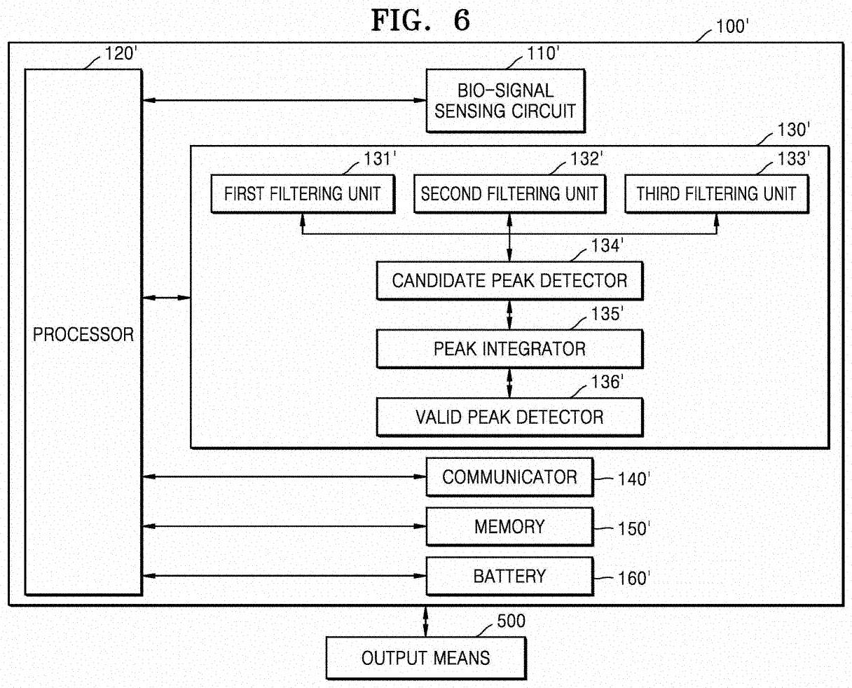

FIG. 5A is a block diagram of the bio-signal measuring apparatus 100 according to one or more embodiments of the present disclosure. FIG. 5B is a block diagram of the filtering signal divided into windows according to one or more embodiments of the present disclosure. FIG. 6 is a block diagram of another bio-signal measuring apparatus 100′ according to one or more embodiments.

The bio-signal measuring apparatus 100, 100′ may be a computing device including one or more processors and may perform the method of processing an electrocardiogram signal by using the one or more processors. The bio-signal measuring apparatus may further include a memory. The memory may be electrically connected to the electrocardiogram signal processing device or may be connected to the electrocardiogram signal processing device through a network. Here, the electrocardiogram signal may include one or more waveforms. For reference, the method of detecting a valid peak of an electrocardiogram signal, according to one or more embodiments of the present disclosure, is performed on a preset detection region. In addition, the electrocardiogram signal used in the embodiments of the present disclosure is also referred to as a QRS signal. The bio-signal measuring apparatus 100 includes a bio-signal sensing circuit 110, a processor 120, a communication 140, a memory 150, a battery 160, and a signal processing unit 130. The signal processing unit 130 includes a first filtering unit 131, a second filtering unit 132, a candidate peak detector 134, a peak integrator 135, and a valid peak detector 136. At least one of the first filtering unit 131, the second filtering unit 132, the candidate peak detector 134, the peak integrator 135, and the valid peak detector 136 may be implemented as a hardware component. At least one of the first filtering unit 131, the second filtering unit 132, the candidate peak detector 134, the peak integrator 135, and the valid peak detector 136 may be implemented as software code and executed by a separately provided processor. In some forms, the bio-signal sensing circuit 110, the first filtering unit 131, the second filtering unit 132, the candidate peak detector 134, the peak integrator 135, and the valid peak detector 136 may be provided and implemented in one apparatus, or in other forms, may be provided in two or more apparatuses.

The bio-signal measuring apparatus 100 may process a bio-signal sensed via the bio-signal sensing circuit 110. The bio-signal sensing circuit 110 may be invasively or non-invasively attached to the body of a user and sense a bio-signal of the user. The bio-signal sensing circuit 110 may acquire an electrocardiogram signal that is an electrical change according to a heartbeat obtained by an electrode. Although not shown in FIG. 5A, the bio-signal sensing circuit 110 may include a plurality of measurement electrodes for acquiring an electrocardiogram signal as shown in FIG. 4A and an AD conversion unit for converting, into digital electrocardiogram data, an analog electrocardiogram signal measured by the measurement electrode. The bio-signal sensing circuit 110 may further include an amplification unit for amplifying an electrocardiogram signal.

The processor 120 may receive and store the electrocardiogram signal in the memory 150. The processor 120 may transmit the electrocardiogram signal to the first filtering unit 131 and/or the second filtering unit 132 and control to generate filtered signals. Signals filtered under the control of the processor 120 may be sequentially processed through the candidate peak detector 134, the peak integrator 135, and the valid peak detector 136.

The communicator 140 may transmit the electrocardiogram signal, information on generated valid peaks, and the like to an output means 500 or an external electronic device. The communicator 140 may receive data from an output means 500 or an external electronic device.

The memory 150 may store data necessary for detecting a valid peak, data on the detected valid peak, data on the candidate peaks, etc. The memory 150 may be a random access memory (RAM) such as dynamic random access memory (DRAM) and static random access memory (SRAM), a read-only memory (ROM), and an electrically erasable programmable read-only memory (EEPROM). It can be implemented in different types.

The battery 160 may supply power to the bio-signal sensing circuit 110, the processor 120, the communicator 140, the memory 150.

The signal processing unit 130 may include the first filtering unit 131 and the second filtering unit 132 for filtering a bio-signal into determined frequency bands, and may include the candidate peak detector 134, the peak integrator 135, and the valid peak detector 136 for detecting peaks included in filtered signals. The signal processing unit 130 may be a software implemented and recorded recording medium. The signal processing unit 130 may receive the electrocardiogram signal from the bio-signal sensing circuit 110 or load the electrocardiogram signal in the memory 150. The electrocardiogram signal may be digitally converted in the bio-signal sensing circuit 110.

The electrocardiogram signal is filtered into a first frequency band via the first filtering unit 131, and an electrocardiogram signal is filtered into a second frequency band via the second filtering unit 132. Here, for convenience of description, frequency conversion is used, but various types of conversion methods (e.g., wavelet conversion, Hilbert conversion, and the like) may be used.

The candidate peak detector 134 detects first candidate peaks included in a first filtering signal filtered by the first filtering unit 131. The candidate peak detector 134 detects second candidate peaks included in a second filtering signal filtered by the second filtering unit 132. The candidate peak detector 134 detects candidate peaks on the basis of QRS template.

The peak integrator 135 calculates average peak time values of the first candidate peaks of the first filtering signal and the second candidate peaks of the second filtering signal by using windows defined by time intervals. Here, a window may be set to have a width of a preset unit time. Here, the width (size) of the preset unit time may be determined on the basis of a heart rate of a user or an R-R interval. As shown in FIG. 5B, the first or the second filtering signal is divided into windows W1, W2, W3, W4, . . . Wn−1, Wn having the width of the preset unit time by way of one example.

The peak integrator 135 may merge the first candidate peaks with the second candidate peaks on the basis of time values and calculate, as an average peak time value, an average value of time values of the first candidate peaks and the second candidate peaks by using windows having determined sizes after the mergence. For example, when a first window includes a candidate peak A and a candidate peak B, an average value of a time value of the candidate peak A and a time value of the candidate peak B may be converted into an average peak time value. When the first window does not include candidate peaks, an average peak time value corresponding to the first window is recorded as nope and is not calculated.

In the above method, the peak integrator 135 may calculate average peak time values while moving, along the x axis, a window having a determined size in data including first candidate peaks and second candidate peaks.

The valid peak detector 136 calculates, in the first filtering signal and the second filtering signal, complexity values for time intervals including peak time values and detects a valid peak point on the basis of the complexity value in the first filtering signal and the complexity value in the second filtering signal. Here, a complexity value may be calculated by using a complexity calculation algorithm such as Shannon entropy or the like but is not limited thereto. The complexity value may be calculated in various methods such as Kolmogorov, monotone, prefix, and decision. The complexity value indicates the degree of complexity of the values generated within the window, and appears higher as the distribution of the signal becomes more irregular. For instance, in the window Wn of FIG. 5B, the maximum value of the signal is not regular, so the complexity value can be calculated high.

The complexity value may be determined on the basis of an occurrence frequency for each magnitude section of measured values of pieces of data of a measured bio-signal. Measured values of a signal may be divided into a plurality of magnitude bins on the basis of the magnitude thereof. Accordingly, a first occurrence frequency may be calculated by extracting data (points) of a signal included in a first magnitude bin. A high occurrence frequency in one magnitude bin may indicate that signals (data) are generated in a similar pattern, and a low occurrence frequency in each magnitude bin may indicate that signals have irregular patterns, i.e., complexity forms.

In other words, a complexity value may be calculated as in Equation 1 below by defining a magnitude bin generated on the basis of a value obtained by measuring a signal and calculating an occurrence frequency of the number of data points in each of magnitude bins.

$$SE = - {{\sum }_{m = 1}}^{M} p ( m ) {log}_{2} p ( m ) \qquad [ Equation 1 ]$$

wherein M is a bin magnitude value of the magnitude of a signal, and p(m) is an occurrence probability function of the signal in each bin interval. For example, when m is 1, p(m) may represent a ratio between the total number L of data points and the number N of data points corresponding to m=1.

$$p ( m ) = \frac{N}{L}$$

In another embodiment, a complexity value may be calculated by using a turning point ratio (TRR). In this case, when the number of turning points per unit time is great, a complexity value may be calculated high. When the number of turning points per unit time is less, a complexity value may be calculated to be low. A TRR may be a value for identifying a change in magnitude of two adjacent pieces of data at a particular point in time and representing a ratio of a turning point to the total length of data. Here, the turning point refers to a point at which the tangent of a signal is 0, i.e., a point at which a direction of a value of data is changed. A complexity value may be calculated as in Equation 2 below:

$$TPR = \frac{N ( ( {x}_{i} - {x}_{i - 1} ) ( {x}_{i} - {x}_{i + 1} ) > 0 )}{L} \qquad [ Equation 2 ]$$

wherein N (x) denotes the number of pieces of data satisfying condition x, and L denotes the magnitude of a data section (N((xᵢ−xᵢ₋₁)(xᵢ−xᵢ₊₁)>0)) denotes the number of points (data) that is (xᵢ−xᵢ₋₁)(xᵢ−xᵢ₊₁)>0) in the section L, i.e., the number of turning points.

The valid peak detector 136 determines a first time interval including a first average peak time value, calculates a first complexity value of a first filtering for the first time interval, calculates a second complexity value of a second filtering signal for the first time interval, and, when the first complexity value and the second complexity value are detected to exceed a preset threshold complexity level, calculates a first peak time value as a peak occurrence time value. The valid peak detector 136 may detect, through the above process, peak occurrence time values in time intervals.

When the first complexity value, the second complexity value or both are detected not to exceed the preset threshold complexity level, the first peak time value does not become the peak occurrence time value.

The valid peak detector 136 may detect valid peaks in an electrocardiogram signal by using peak occurrence time values.

As used herein, a threshold complexity level that is a basis for selecting a peak occurrence time value may be determined through a process of performing machine learning by using, as input data, a complexity value of a time interval including peaks and using, as output data, whether or not a peak is valid but is not limited thereto. The threshold complexity level may be determined by using various types of neural networks, statistical techniques, or the like which are available in the pertinent art.

As shown in FIG. 5B, the first or the second filtering signal is divided into windows W1, W2, W3, . . . Wn−1, Wn. The complexity value indicates the degree of complexity of the values generated within the window W1, W2, W3, . . . Wn−1, Wn.

As shown in FIG. 6, peak occurrence time values may be detected by using signals filtered via a first filtering unit, a second filtering unit, and a third filtering unit to detect valid peaks. The bio-signal measuring apparatus 100′ of FIG. 6 may be implemented by including a bio-signal sensing circuit 110′, a processor 120′, a communication 140′, a memory 150′), a battery 160′, and a signal processing unit 130′. The signal processing unit 130′ includes a first filtering unit 131′, a second filtering unit 132′, and a third filtering unit 133′. To the extent that the bio-signal sensing circuit 110′ is the same as the structure of the bio-signal sensing circuit 110, a detailed description thereof will be omitted herein.

At least one of the first filtering unit 131′, the second filtering unit 132′, the third filtering unit 133′, a candidate peak detector 134′, a peak integrator 135′, and a valid peak detector 136′ may be implemented as a hardware component. At least one of the first filtering unit 131′, the second filtering unit 132′, the third filtering unit 133′, the candidate peak detector 134′, the peak integrator 135′, and the valid peak detector 136′ may be implemented as software code and executed by a separately provided processor. The first filtering unit 131′, the second filtering unit 132′, the third filtering unit 133′, the candidate peak detector 134′, the peak integrator 135′, and the valid peak detector 136′ may be implemented in one apparatus or may be implemented in two or more apparatuses.

The signal processing unit 130′ may be implemented as remote apparatuses to receive a bio signal from the bio-signal sensing circuit 110′ and process the bio-signal. The signal processing unit 130′ may receive the electrocardiogram signal from the bio-signal sensing circuit 110′ or load the electrocardiogram signal in the memory 150′. The electrocardiogram signal may be digitally converted in the bio-signal sensing circuit 110′.

The candidate peak detector 134′ detects first candidate peaks included in a first filtering signal filtered by the first filtering unit 131′, second candidate peaks included in a second filtering signal filtered by the second filtering unit 132′, and third candidate peaks included in a third filtering signal filtered by the third filtering unit 133′. The candidate peak detector 134′ may generate, as one integrated piece of data, the first candidate peaks, the second candidate peaks, and the third candidate peaks.

The peak integrator 135′ calculates average peak time values of the first candidate peaks of the first filtering signal, the second candidate peaks of the second filtering signal, and the third candidate peaks of the third filtering signal by using windows defined by time intervals. An average of time values of a 1-1^{th} candidate peak, a 1-2^{th} candidate peak, and a 1-3^{th} candidate peak included in a first window defined by a first time interval (e.g., W₁ in FIG. 5B) may be calculated and set as an average peak time value. The 1-1^{th} candidate peak, the 1-2^{th} candidate peak, and the 1-3^{th} candidate peak may belong to at least one of first candidate peaks, second candidate peaks, and third candidate peaks. The 1-1^{th} candidate peak, the 1-2^{th} candidate peak, and the 1-3^{th} candidate peak may be included in different filtering signals, respectively. The 1-1^{th} candidate peak and the 1-2^{th} candidate peak may be included in a first filtering signal or the 1-1^{th} candidate peak, the 1-2^{th} candidate peak, and the 1-3^{th} candidate peak may be included in the first filtering signal.

The peak integrator 135′ may merge first candidate peaks, second candidate peaks, and third candidate peaks on the basis of time values and calculate, as an average peak time value, an average value of time values of the first candidate peaks, the second candidate peaks, and the third candidate peaks by using windows having determined time intervals (magnitudes) after the mergence. The number of candidate peaks included in a window may be 0 or may be three or more. For example, when a first window includes a candidate peak A, a candidate peak B, a candidate peak C, and a candidate peak D, an average value of time values of the candidate peaks A, B, C, and D may be converted into an average peak time value. When the first window does not include candidate peaks, an average peak time value corresponding to the first window is recorded as nope and is not calculated.

In the above method, the peak integrator 135′ may calculate average peak time values while moving, along the x axis, a window having a determined size in data including first candidate peaks and second candidate peaks.

The valid peak detector 136′ calculates, in first through third filtering signals, complexity values for time intervals including peak time values, respectively, and detects at least one peak occurrence time value among average peak time values on the basis of the respectively calculated complexity values. Here, a complexity value may be calculated by using a complexity calculation algorithm such as Shannon entropy, Sample entropy or Turning point ratio.

The valid peak detector 136′ determines a first time interval including a first average peak time value, calculates a first complexity value of a first filtering signal, a second complexity value of a second filtering signal, and a third complexity value of a third filtering signal for the first time interval, calculates a second complexity value of a second filtering signal for the first time interval, and, when the first complexity value and the second complexity value are detected to exceed a preset threshold complexity level, detects a first peak time value as a peak occurrence time value. The valid peak detector 136′ may detect valid peaks on the basis of peak occurrence time values calculated through the above process. When at least one of the first complexity value, the second complexity value, and the third complexity value is detected not to exceed the preset threshold complexity level, the first average peak time value does not become the peak occurrence time value.

The valid peak detector 136′ may detect valid peaks in an electrocardiogram signal by using peak occurrence time values.

The processor 120′ may receive and store the electrocardiogram signal and record in the memory 150′. The processor 120′ may transmit the electrocardiogram signal to the first filtering unit 131′, the second filtering unit 132′ and/or the third filtering unit 133′ and control to generate filtered signals. Signals filtered under the control of the processor 120′ may be sequentially processed through the candidate peak detector 134′, the peak integrator 135′, and the valid peak detector 136′.

The communicator 140′ may transmit the electrocardiogram signal, information on generated valid peaks, and the like to an external electronic device. The communicator 140′ may receive data from an external electronic device.

The memory 150′ may store data necessary for detecting a valid peak, data on the detected valid peak, data on the candidate peaks, and the like. The memory 150′ may be a random access memory (RAM) such as dynamic random access memory (DRAM) and static random access memory (SRAM), a read-only memory (ROM), and an electrically erasable programmable read-only memory (EEPROM). It can be implemented in different types. The battery 160′ may supply power to the bio-signal sensing circuit 110′, the processor 120′, the communicator 140′, the memory 150′.

The bio-signal measuring apparatus 100 or 100′ may generate data on an electrocardiogram signal, data on a signal filtered from the electrocardiogram signal, data on a valid peak detected in the electrocardiogram signal, and the like. The data on the valid peak may include a time interval in which the valid peak is generated, a window in which the valid peak is generated and time values in which the valid peak is generated, and data values of the valid peak.

The bio-signal measuring apparatus 100, 100′ may output an electrocardiogram signal, data displaying valid peaks corresponding to the electrocardiogram signal, and information on valid peaks (the time interval in which the valid peak occurred, the window, the time value, etc.) data can be generated. The bio-signal measuring apparatus 100 and 100′ may transmit output data to an external output device or electronic device so that the electrocardiogram signal and at least one peak included in the electrocardiogram signal are visually displayed. In some embodiments, the electrocardiogram signal having the valid peak is displayed and an electrocardiogram signal having a peak that does not appear during the time duration (TD) may be disregarded or not displayed.

The bio-signal measuring apparatus 100 or 100′ is capable of processing the electrocardiogram signal and accurately determining valid peaks of the electrocardiogram signal including an R-peak of the electrocardiogram signal and solving the technical problems described above.

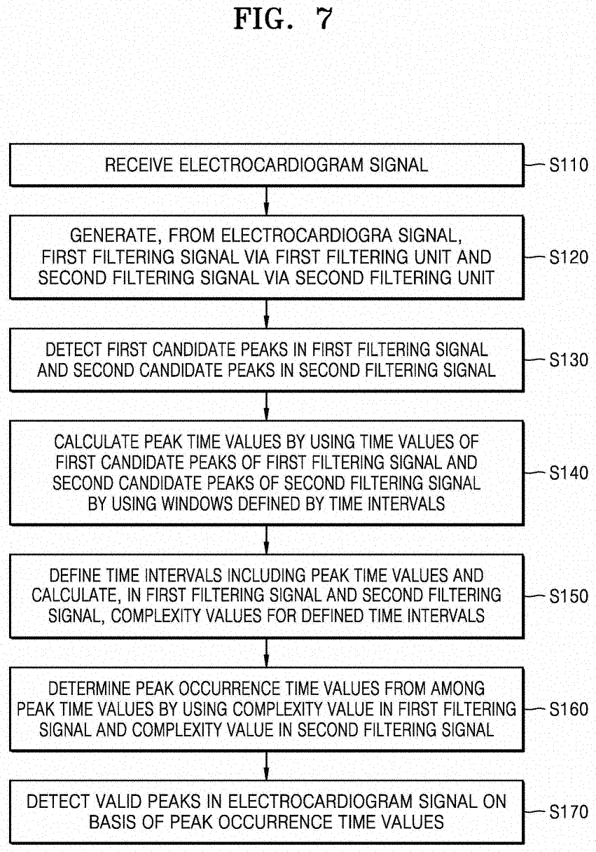

FIG. 7 is a flowchart of a method of detecting signal peaks according to one or more embodiments.

In operation S110, the bio-signal measuring apparatus 100 receives an electrocardiogram signal from a sensing circuit. In operation S120, the bio-signal measuring apparatus 100 filters the electrocardiogram signal into a first frequency band via the first filtering unit 131 and filters the electrocardiogram signal into a second frequency band via the second filtering unit 132.

In operation S130, the bio-signal measuring apparatus 100 detects first candidate peaks included in a first filtering signal filtered by the first filtering unit 131. The bio-signal measuring apparatus 100 detects second candidate peaks included in a second filtering signal filtered by the second filtering unit 132.

In operation S140, the bio-signal measuring apparatus 100 calculates average peak time values based on time values of the first candidate peaks of the first filtering signal and time values of the second candidate peaks of the second filtering signal by using windows defined as time intervals. The average peak time values may be calculated as a set of average values of time value of the first candidate peak and time value of the second candidate peak included in each window. Here, a window may be set to have a width of a preset unit time. Here, the width (magnitude) of the unit time may be determined on the basis of a heartbeat of a user or an R-R interval.

The bio-signal measuring apparatus 100 may merge the first candidate peaks with the second candidate peaks on the basis of time values and calculate, as an average peak time value, an average value of time values of the first candidate peaks and the second candidate peaks by using windows having a determined size after the mergence. When a determined first window does not include candidate peaks, an average peak time value corresponding to the first window is recorded as nope and is not calculated.

The bio-signal measuring apparatus 100 may calculate average peak time values of each window while moving, along the x axis, the window including the first candidate peaks and the second candidate peaks.

In operation S150, the bio-signal measuring apparatus 100 calculates, in the first filtering signal and the second filtering signal, complexity values for time intervals including the peak time values and detects a valid peak point on the basis of the complexity value in the first filtering signal and the complexity value in the second filtering signal. Here, a complexity value may be calculated by using a complexity calculation algorithm such as Shannon entropy.

The complexity value may be determined on the basis of an occurrence frequency for each magnitude section of measured values of pieces of data of a measured bio-signal. Measured values of a signal may be divided into a plurality of magnitude bins on the basis of the magnitude thereof. Accordingly, a first occurrence frequency may be calculated by extracting pieces of data (points) of a signal included in a first magnitude bin. A high occurrence frequency in one magnitude bin may indicate that signals (data) are generated in a similar pattern, and a low occurrence frequency in each magnitude bin may indicate that signals have irregular patterns, i.e., complex forms.

In other words, a complexity value may be calculated as in Equation 1 by defining magnitude bins generated on the basis of values obtained by measuring a signal and calculating an occurrence frequency of the number of data points in each of the magnitude bins.

In another embodiment, a complexity value may be calculated by using a turning point ratio (TPR). In this case, when the number of turning points per unit time is great, a complexity value may be calculated high. When the number of turning points per unit time is small, a complexity value may be calculated to be low. The TPR may be a value for identifying a change in magnitude of adjacent pieces of data at a particular point in time and representing a ratio of a turning point to the total length of data. Here, a turning point refers to a point at which the tangent of a signal is 0, i.e., a point at which a direction of a value of data is changed. A complexity value may be calculated as in Equation 2.

The bio-signal measuring apparatus 100 determines a first time interval including a first average peak time value, calculates a first complexity value of a first filtering signal for the first time interval, calculates a second complexity value of a second filtering signal for the first time interval, and, when the first complexity value and the second complexity value are detected to exceed a preset threshold complexity level, detects a first average peak time value as a peak occurrence time value.

In operation S160 (see FIG. 7), the bio-signal measuring apparatus 100 may determine peak occurrence time values among average peak time values through the above process.

In operation S170, the bio-signal measuring apparatus 100 may detect valid peaks in an electrocardiogram signal by using the peak occurrence time values. The bio-signal measuring apparatus 100 is capable of processing the electrocardiogram signal and accurately determining valid peaks of the electrocardiogram signal including an R-peak of the electrocardiogram signal.

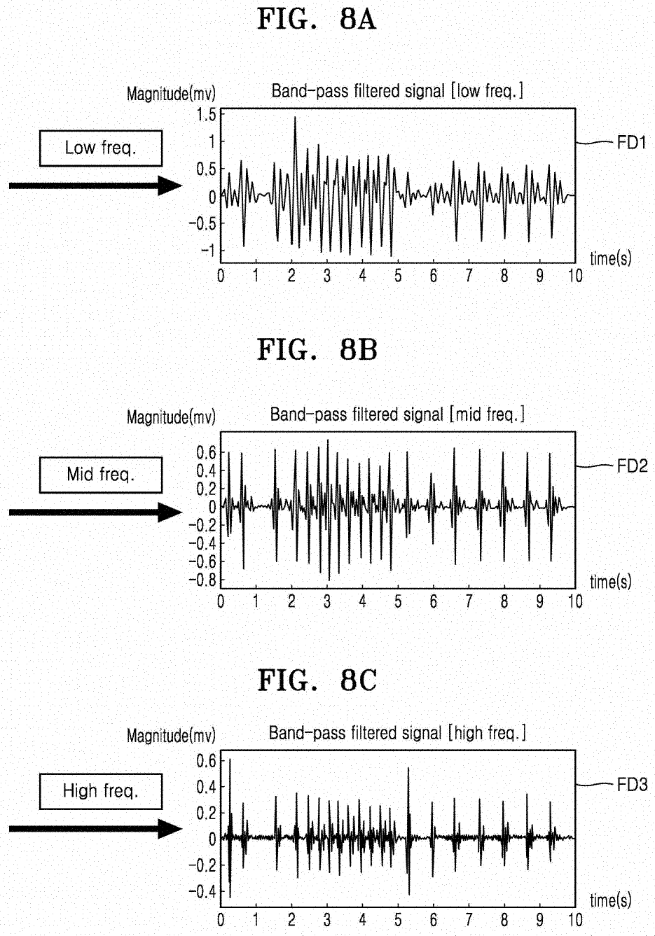

FIG. 8A is an exemplary view illustrating a first filtering signal FD1, FIG. 8B is an exemplary view illustrating a second filtering signal FD2, and FIG. 8C is an exemplary view illustrating a third filtering signal FD3. The bio-signal measuring apparatus 100 may generate the first filtering signal FD1 by frequency-transforming with first frequency. The bio-signal measuring apparatus 100 may generate the second filtering signal FD2 by frequency-transforming with second frequency. The bio-signal measuring apparatus 100 may generate the third filtering signal FD3 by frequency-transforming with third frequency.

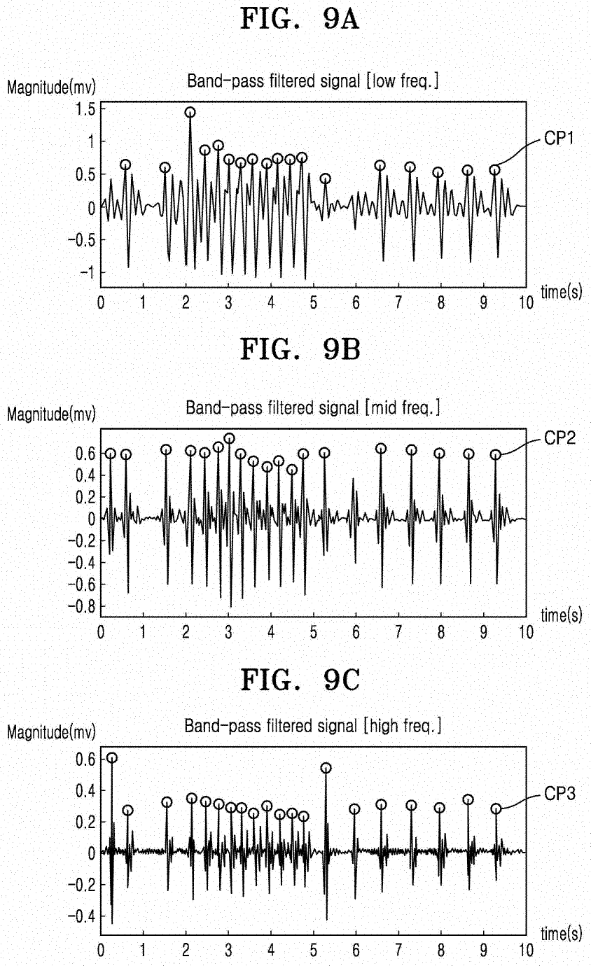

FIG. 9A is an example view illustrating first candidate peaks CP1, FIG. 9B is an example view illustrating second candidate peaks CP2, and FIG. 9C is an example view illustrating third candidate peaks CP3. The bio-signal measuring apparatus 100 may detect candidate peaks from first, second and third filtering signals. The bio-signal measuring apparatus 100 may detect first candidate peaks CP1 in the first filtering signal by using a form characteristic, a slope value, a threshold voltage value, an R-R interval, and the like of the first filtering signal. The bio-signal measuring apparatus 100 may detect second candidate peaks CP2 in the second filtering signal by using a form characteristic, a slope value, a threshold voltage value, an R-R interval, and the like of the second filtering signal. The bio-signal measuring apparatus 100 may detect third candidate peaks CP3 in the third filtering signal by using a form characteristic, a slope value, a threshold voltage value, an R-R interval, and the like of the third filtering signal.

The bio-signal measuring apparatus 100 may detect candidate peaks in a filtering signal by using a form characteristic, a slope value, a threshold voltage value, an R-R interval, and the like of the filtering signal.

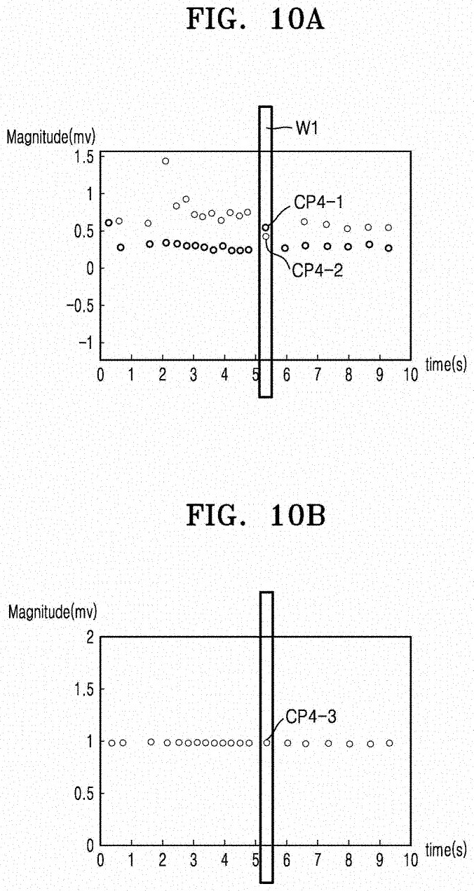

FIG. 10A is an exemplary diagram of candidate peaks detected according to one or more embodiments. FIG. 10B is a view for explaining a process of determining an average peak time value, according to one or more embodiments.

As shown in FIG. 10A, one or more candidate peaks CP4-1 and CP4-2 included in a window W1 defined by a certain time interval may be extracted, and an average of time values of the candidate peaks CP4-1 and CP4-2 may be calculated as an average peak time value. As shown in FIG. 10B, a point corresponding to the average peak time value calculated from the candidate peaks CP4-1 and CP4-2 may be converted into CP4-3.

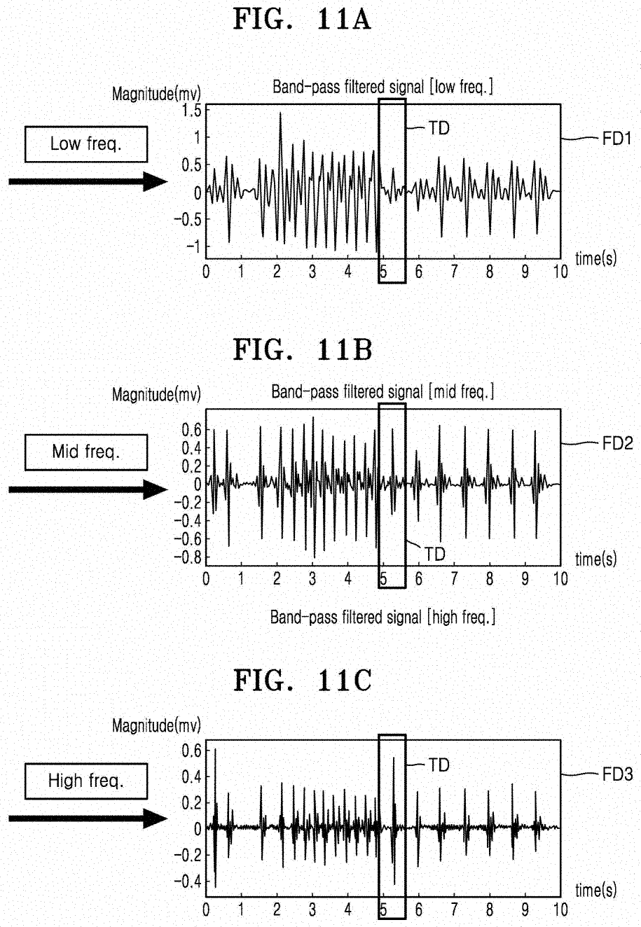

FIG. 11A is a diagram of a first filtering signal filtered to a first frequency band according to one or more embodiments. FIG. 11B is a diagram of a second filtering signal filtered to a second frequency band according to one or more embodiments. FIG. 11C is a diagram of a third filtering signal filtered to a third frequency band according to one or more embodiments. As shown in FIG. 11A, FIG. 11B, and FIG. 11C, a time interval TD including an average of time values of the candidate peaks may be defined, and complexity values for the defined time interval TD may be calculated from the first through the third filtering signals, respectively. A first complexity value in the TD may be calculated from the first filtering signal. A second complexity value in the TD may be calculated from the second filtering signal. A third complexity value in the TD may be calculated from the third filtering signal.

When all of the complexity values for the time interval TD exceed a preset threshold complexity level, the time value of CP4-3 included in the time interval TD may be determined as a peak occurrence time value. When the first complexity value, the second complexity value, and the third complexity value all exceed the threshold complexity level, the time value of CP4-3 may be classified as a peak occurrence time value.

Accordingly, according to one or more embodiments, the bio-signal measuring apparatus 100 may determine peak occurrence time values of peak points in an electrocardiogram signal.

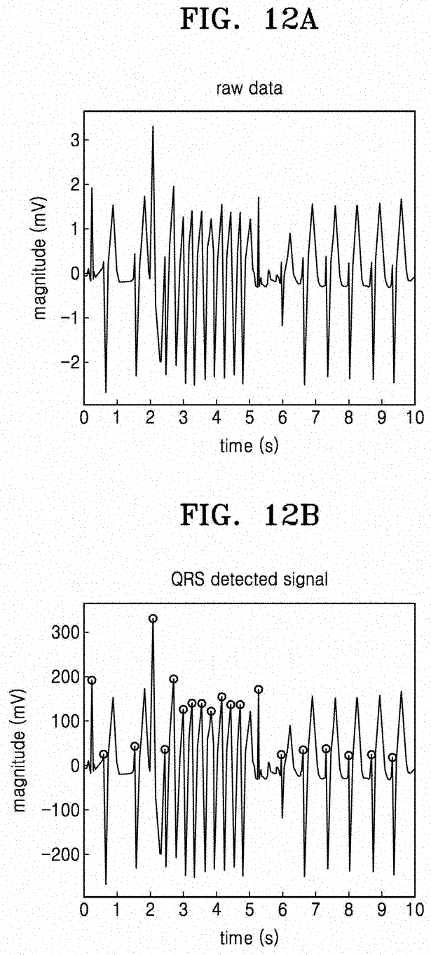

FIG. 12A is an example view illustrating an electrocardiogram signal and FIG. 12B is an example view illustrating an electrocardiogram signal from which signal peaks are detected, according to one or more embodiments.

As shown in FIG. 12A and FIG. 12B, the measured electrocardiogram signal may be converted into data including the detected peak data. The bio-signal measuring apparatus may generate output data in which points corresponding to peaks in the ECG signal are displayed as separate marks. As shown in FIG. 12A, according to embodiments of the present disclosure, effective peaks of a waveform may be detected using an electrocardiogram signal measured with one electrode by way of example.

The apparatus and the method described above enable processing of an electrocardiogram signal and accurate determination of valid peaks of the electrocardiogram signal including an R-peak of the electrocardiogram signal and solving the technical problems in the related art.

The apparatus described above may be embodied as hardware components, software components, and/or a combination of hardware components and software components. For example, the apparatus and components described in the embodiments may be embodied by using one or more general purpose computers or special purpose computers such as a processor, a controller, an arithmetic logic unit (ALU), a digital signal processor, a microcomputer, a field programmable gate array (FPGA), a programmable logic unit (PLU), a microprocessor, or any other apparatuses capable of executing and responding to instructions. A processing apparatus may execute an operating system (OS) and one or more software applications executed on the OS. In addition, the processing apparatus may access, store, manipulate, process, and generate data in response to the execution of software. For convenience of description, one processing apparatus may be described as being used, but one of ordinary skill in the art may understand that the processing apparatus may include a plurality of processing elements and/or a plurality of types of processing elements. For example, the processing apparatus may include a plurality of processors or one processor and one controller. Also, the processing apparatus may include another processing configuration such as a parallel processor.

Software may include computer program, code, instructions, or a combination of one or more thereof, and configure the processing apparatus to operate as wanted or independently or collectively instruct the processing apparatus. Software and/or data may be permanently or temporarily embodied in any type of machine, a component, a physical device, virtual equipment, a computer storage medium or device, or transmitted signal waves to be interpreted by the processing apparatus or to provide the processing apparatus with instructions or data. The software may be distributed over networked computer systems to be stored or executed in a distributed manner. The software and data may be stored in one or more computer-readable recording media.

The method according to the embodiments may be embodied in the form of program instructions that may be executed through various types of computer means and then recorded in a computer-readable recording medium. The computer-readable recording medium may include program instructions, data files, data structures, and the like alone or in combination. The program instructions recorded on the computer-readable recording medium may be particularly designed and configured for the embodiments or may be well known to and used by one of ordinary skill in the computer software art. Examples of the computer-readable recording medium include magnetic media such as a hard disk, a floppy disk, and a magnetic tape, optical media such as a CD-ROM and a DVD, magneto-optical media such as a floptical disk, and a hardware device particularly configured to store and execute program instructions such as ROM, RAM, flash memory, and the like. Examples of the program instructions include not only machine language code generated by a compiler, but also high-level language code that may be executed by a computer by using an interpreter or the like. The hardware device described above may be configured to operate as one or more software modules so as to perform operations of the embodiments, and the reverse thereof is the same.

According to one or more embodiments, signal peaks may be detected by using a complexity value of a signal around a peak.

In addition, according to one or more embodiments, signal peaks in a time interval having a complexity value exceeding a determined threshold complexity level may be detected via machine learning or an artificial intelligence algorithm.

Although the embodiments have been described above by limited embodiments and drawings, various modifications and changes may be made from the above description by one of ordinary skill in the art. For example, the described techniques may be performed in a different order than the described method, and/or components of the described system, structure, device, circuit, and the like may be combined or joined in a different form than the described method, or even if replaced or substituted by other components or equivalents, and an appropriate result may be achieved.

Therefore, other embodiments, other aspects, and equivalents to claims also belong to the scope of claims that will be described below.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A wearable device, comprising:
a biosignal sensing circuit including a plurality of electrodes positioned at a plurality of different locations on a body of an object and configured to sense an electrocardiogram signal and convert the electrocardiogram signal digitally;
a processor coupled to the biosignal sensing circuit and operable to receive the electrocardiogram signal from the biosignal sensing circuit; and
a memory that stores the electrocardiogram signal and stores executable instructions that, when executed by the processor, facilitate performance of operations, the operations comprising:
generating a first filtered signal by filtering the electrocardiogram signal in a first frequency band,
generating a second filtered signal by filtering the electrocardiogram signal in a second frequency band,
detecting a plurality of peaks in the first filtered signal as first candidate peaks based on a magnitude of the first filtered signal,
detecting a plurality of peaks in the second filtered signal as second candidate peaks based on a magnitude of the second filtered signal,
during each preset time window having a predetermined time interval, determining an average peak time value based on a first candidate peak in the first filtered signal and a second candidate peak in the second filtered signal;
determining a time duration (TD) based on the average peak time value;
determining a first complexity value of the first filtered signal and a second complexity value of the second filtered signal in the time duration (TD), the first complexity value and the second complexity value indicative of an occurrence frequency pattern of the plurality of peaks in the first filtered signal and in the second filtered signal, respectively;
determining whether or not both the first complexity value and the second complexity value exceed a preset threshold complexity level, and
upon determination that both the first complexity value and the second complexity value exceed the preset threshold complexity level, determining a peak appearing during the time duration (TD) as a valid peak of the electrocardiogram signal; and
a display coupled to the processor and operable to display the electrocardiogram signal having the valid peak and disregard an electrocardiogram signal having a peak that does not appear during the time duration (TD).

2. The wearable device of claim 1, wherein the first or the second complexity value is calculated by one of Shannon entropy, Sample entropy, and Turning point ratio.

3. The wearable device of claim 1, wherein the operations further comprise determining the preset threshold complexity level based on complexity values of time intervals including R peaks of electrocardiogram signals measured in the past.

4. The wearable device of claim 1, wherein the determining the first complexity value and the second complexity value further comprises determining the first complexity value or the second complexity value based on the occurrence frequency pattern of each magnitude section of the sensed electrocardiogram signal.

5. The wearable device of claim 1, wherein the operations further comprise forgoing to determine the average peak time value when no candidate peak is present during a preset time window.

6. A wearable device, comprising:
a biosignal sensing circuit including a plurality of electrodes positioned at a plurality of different locations on a body of an object and configured to sense an electrocardiogram signal and convert the electrocardiogram signal digitally;
a processor coupled to the biosignal sensing circuit and operable to receive the electrocardiogram signal from the biosignal sensing circuit; and
a signal processing unit coupled to the processor and including:
a first filtering unit and a second filtering unit;
a candidate peak detector coupled to the first filtering unit and the second filtering unit;
a peak integrator coupled to the candidate peak detector;
a valid peak detector coupled to the peak integrator; and
output mean coupled to the valid peak detector;
a display coupled to the processor and operable to display the electrocardiogram signal;
wherein the signal processing unit, when executed by the processor, facilitate performance of operations, the operations comprising:
generating, with the first filtering unit, a first filtered signal by filtering the electrocardiogram signal in a first frequency band,
generating, with the second filtering unit, a second filtered signal by filtering the electrocardiogram signal in a second frequency band,
detecting, with the candidate peak detector, a plurality of peaks in the first filtered signal as first candidate peaks based on a magnitude of the first filtered signal,
detecting, with the candidate peak detector, a plurality of peaks in the second filtered signal as second candidate peaks based on a magnitude of the second filtered signal, during each preset time window having a predetermined time interval,
determining, with the peak integrator, an average peak time value based on a first candidate peak in the first filtered signal and a second candidate peak in the second filtered signal;
determining, with the valid peak detector, a time duration (TD) based on the average peak time value;
determining, with the valid peak detector, a first complexity value of the first filtered signal and a second complexity value of the second filtered signal in the time duration (TD), the first complexity value and the second complexity value indicative of an occurrence frequency pattern of the plurality of peaks in the first filtered signal and in the second filtered signal, respectively;
determining, with the valid peak detector, whether or not both the first complexity value and the second complexity value exceed a preset threshold complexity level, and
upon determination that both the first complexity value and the second complexity value exceed the preset threshold complexity level, determining, with the valid peak detector, a peak appearing during the time duration (TD) as a valid peak of the electrocardiogram signal; and
a display coupled to the processor and operable to display the electrocardiogram signal having the valid peak and disregard an electrocardiogram signal having a peak that does not appear during the time duration (TD).

7. The wearable device of claim 6, wherein the first or the second complexity value is calculated by one of Shannon entropy, Sample entropy, and Turning point ratio.

8. The wearable device of claim 6, wherein the operations further comprise determining the preset threshold complexity level based on complexity values of time intervals including R peaks of electrocardiogram signals measured in the past.

9. The wearable device of claim 6, wherein the determining the first complexity value and the second complexity value further comprises determining the first complexity value or the second complexity value based on the occurrence frequency pattern of each magnitude section of the sensed electrocardiogram signal.

10. The wearable device of claim 6, wherein the operations further comprise forgoing to determine the average peak time value when no candidate peak is present during a preset time window.

11. A method comprising:
sensing an electrocardiogram signal by a bio-signal sensing circuit of the bio-signal measuring apparatus;
converting digitally the electrocardiogram signal by the bio-signal sensing circuit;
storing the electrocardiogram signal in a memory by a processor of a bio-signal measuring apparatus,
generating, by the processor, a first filtered signal by filtering the electrocardiogram signal in a first frequency band,
generating, by the processor, a second filtered signal by filtering the electrocardiogram signal in a second frequency band,
detecting, by the processor, a plurality of peaks in the first filtered signal as first candidate peaks based on a magnitude of the first filtered signal,
detecting, by the processor, a plurality of peaks in the second filtered signal as second candidate peaks based on a magnitude of the second filtered signal,
during each preset time window having a predetermined time interval, determining, by the processor, an average peak time value based on a first candidate peak in the first filtered signal and a second candidate peak in the second filtered signal;
determining, by the processor, a time duration (TD) based on the average peak time value;
determining, by the processor, a first complexity value of the first filtered signal and a second complexity value of the second filtered signal in the time duration (TD), the first complexity value and the second complexity value indicative of an occurrence frequency pattern of the plurality of peaks in the first filtered signal and in the second filtered signal, respectively;
determining, by the processor, whether or not both the first complexity value and the second complexity value exceed a preset threshold complexity level, and
upon determination that both the first complexity value and the second complexity value exceed the preset threshold complexity level, determining, by the processor, a peak appearing during the time duration (TD) as a valid peak of the electrocardiogram signal,
generating, by the processor, output data displaying information on the electrocardiogram signal having the valid peak, and
storing, by the processor, the output data in the memory.

12. The method of claim 11, further comprising transmitting the output data to an external device which displays the electrocardiogram signal having the valid peak based on the output data.

13. The method of claim 11, wherein the first or the second complexity value is calculated by one of Shannon entropy, Sample entropy, Turning point ratio.

14. The method of claim 11, further comprising determining the preset threshold complexity level based on complexity values of time intervals including R peaks of electrocardiogram signals measured in the past.

15. The method of claim 11, wherein the determining the first complexity value and the second complexity value further comprises determining the first complexity value or the second complexity value based on the occurrence frequency pattern of each magnitude section of the sensed electrocardiogram signal.

16. The method of claim 11, further comprising forgoing to determine the average peak time value when no candidate peak is present during a preset time window.

17. A non-transitory computer-readable recording medium storing therein executable instructions that, when executed by a processing system including a processor, facilitate performance of operations as claimed in claim 1.
